(19) United States
(12) Patent Application Publication
(10) Pub. No.: US 2010/0112098 A1
(43) Pub. Date: May 6, 2010

(54) EFFECTS OF A DECAFFEINATED GREEN COFFEE EXTRACT ON BODY WEIGHT CONTROL BY REGULATION OF GLUCOSE METABOLISM

(75) Inventors: Benoit Lemaire, Moulon (FR); Sophie Lafay, Montagne (FR); Karine Nardon, Libourne (FR); Alvin Ibarra, Hoboken, NJ (US); Marc Roller, Morieres Les Avignon (FR); Jacques Dikansky, Avignon (FR)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: Naturex, S.A.

(21) Appl. No.: 12/263,292

(22) Filed: Oct. 31, 2008

(51) Int. Cl. A61K 36/82; A61K 31/235

(52) U.S. Cl. 424/729; 514/533

(57) ABSTRACT

A method of controlling body weight in humans by administering an amount of decaffeinated green coffee extract effective to treat a subject. A preferred green coffee extract contains a ratio of 5-caffeoylquinic acid (5-CQA) and total chlorogenic acids (tCGA) (5-CQA/tCGA) of between about 0.2 and 0.3. More preferably, the concentration of tCGA is greater than about 45% and the concentration of 5-CQA is less than about 10%. A preferred method of administration consists of administering between about 200 mg and about 1,000 mg per day, more preferably administering about 400 mg per day.

### FIELD OF THE INVENTION

The present invention relates to dietary supplements, −+ and more particularly to an extract of green coffee.

### BACKGROUND OF THE INVENTION

The prevalence of obesity (BMI≧30 kg/m²) continues to be a health concern for adults, children and adolescents in the United States. Data from the NHANES survey shows that among adult men the prevalence of obesity increased from 31.1% in 2003-04 to 33.3% in 2005-06, a small but not statistically significant change. Among adult women, the prevalence of obesity increased from 33.2% in 2003-04 to 35.3% in 2005-06, again a small but not significant change (Ogden et al. Gastroenterology 2007; 132(6):2087-2102). In Europe, the prevalence of obesity has increased by 10 to 40%, depending on the country, over the last 10 years (OMS # 894, 2003). Type 2 diabetes (T2D), often associated with excess weight, affects more than 4% of the world's population, or more than 240 million people (Schmitt, Britta Nutrition 2001; 44).

Recent epidemiological studies have shown the beneficial effect of coffee in terms of prevention of T2D, also known as fatty diabetes. T2D is a dysfunction of the mechanism that regulates blood glucose concentration, resulting in insulin resistance. This insulin resistance is expressed as abnormal and prolonged hyperglycemia. Before resulting in T2D, this hyperglycemia consists of an excess of blood glucose which can metabolize into triglycerides, hence, causing weight gain.

Caffeine consumption of 5 mg/kg/day is known to have a role in insulin resistance (Graham et al. Can. J. Physiol. Pharmacol 2001; 79(7):559-565). Because coffee is the primary dietary source of caffeine, a number of epidemiological studies have been conducted to assess the correlation between the coffee consumption of different Western and Asiatic populations and the risk of occurrence of T2D.

TABLE 1
Summary of studies on the risk of type 2 diabetes as a function of coffee consumption.

| | | | Number of | | Daily | | |
|---|---|---|---|---|---|---|---|
| | Origin of the | Length of | individuals | | consumption | Decrease | |
| References | population studied | the study | monitored | Sex | of coffee | in risk % | P |
| A. Reunanen, 2003 | Finland | 4 years | 19,518 | Mixed | ≧7 cups | 8 | — |
| S. Carlsson, 2004 | Finland | 6 years | 10,652 | Mixed | ≧7 cups | 35 | — |
| J. Tuomilehto, 2004 | Finland | 12 years | 14,629 | Mixed | ≧10 cups | 61 | <0.001 |
| E. E. Agardh, 2004 | Sweden | 4 years | 3,128 | Men | ≧5 cups | 64 | — |
| E. E. Agardh, 2004 | Sweden | 4 years | 4,821 | Women | ≧5 cups | 72 | — |
| A. Rosengren, 2004 | Sweden | 18 years | 1,361 | Women | >6 cups | 43 | — |
| R. M. Van Dam, 2002 | Holland | 10 years | 17,111 | Mixed | ≧7 cups | 50 | <0.0002 |
| A. Isogawa, 2003 | Japan | 5 years | 4,620 | Mixed | Not specified | 59 | <0.001 |
| E. Salazar-Martinez, 2004 | United States | 22 years | 41,934 | Men | ≧6 cups | 64 | <0.001 |
| E. Salazar-Martinez, 2004 | United States | 18 years | 84,276 | Women | ≧6 cups | 25 | <0.001 |

Numerous epidemiological studies, mainly published between 2002 and 2004, have demonstrated that coffee consumption of between 5 and 10 cups per day decreases the risk of developing T2D. Table 1 summarizes these studies conducted over 202,050 people in five countries, showing a reduction in the risk of diabetes of 40%. One of the most relevant studies was reported by Van Dam et al. (The Lancet, 2002,360:1477-1478) on the influence of higher or lower coffee consumption on health. After monitoring 17,111 Dutch people between 30 and 60 years old for 7 years, they clearly established a positive correlation between coffee consumption and a decrease in the risk of T2D. Participants drinking 7 cups of coffee or more per day were half as likely (P=0.0002) than participants drinking 2 cups of coffee or less per day to develop T2D. Therefore, there is a link between high coffee consumption and a decrease in the risk of T2D.

Naismith et al. (Nutr. Metabol. 1970; 12:144-151) studied the effect of coffee consumption on the blood sugar concentration. Their study, carried out on twenty healthy volunteers, concluded that certain compounds, other than caffeine, significantly reduce fasting blood sugar levels. This was also suggested by Isogawa et al. (The Lancet, Feb. 2003; 361:702-704). They converted the number of cups consumed into the quantity of caffeine ingested and showed that, despite the tendency to decrease the prevalence of fasting hyperglycemia, the consumption of caffeine alone had no notable effect (p=0.012). This study shows that the risk of fasting hyperglycemia is clearly lower in people consuming coffee, compared with its prevalence in tea drinkers, whatever type of preparation-green tea, fermented tea or oolong tea. No significant correlation has been established between the prevalence of fasting hyperglycemia and the consumption of tea, whether in terms of frequency of consumption or quantity of caffeine ingested. Salazar-Martinez (Ann Intern Med. 2004 Jan. 6; 140(1):1-8) concluded that caffeine is not the active substance decreasing the risk of T2D. Indeed, a net decrease in the risk of T2D occurs for consumers of more than 6 cups of coffee per day. The investigators therefore concluded that molecules contained in coffee, but not caffeine alone, have a beneficial effect in terms preventing fasting hyperglycemia.

While caffeine is not the active substance that prevents blood sugar disorders, the various authors of the epidemiological studies mentioned in Table 1 suggest or agree that chlorogenic acids (CGA) do play a highly influential role in this. Current scientific consensus attributes the protective effect of chlorogenic acids to their capacity to regulate postprandial blood sugar concentration, inhibit the intestinal absorption of glucose, improve glucose tolerance, and, to a lesser extent, modulate serum lipid concentrations.

Chlorogenic acids (CGA) are a family of esters formed between certain hydroxycinnamic acids (i.e. caffeic and feluric acids) and (−)-quinic acid. Green (or raw) coffee is a major source of CGA in nature (5-12 g/100 g) (Farah et al. Braz J Plant Physiol. 365 2006; 18:23-36). The major CGA in green coffee are 3-, 4- and 5-caffeoylquinic acids (3-, 4- and 5-CQA), 3,4-, 3,5- and 4,5-dicaffeoylquinic acids (3,4-, 3,5-, and 4,5-diCQA); 3-, 4- and 5-feruloylquinic acids (3-, 4- and 5-FQA) and 3-, 4- and 5-p-coumaroylqunic acids (3-, 4-, and 5-p-CoQA). Caffeoylferuloylquinic acids (CFQA) are minor CGA compounds also found in green coffee, especially in Coffea robusta species. Very small amounts of CGA lactones formed by heating during primary processing may also be observed (Farah et al. Braz J Plant Physiol. 2006, 18:23-36.—Farah et al. J Agric Food Chem. 2005; 53:1505-13).

Coffee berries, which contain the coffee bean, are produced by several species of small evergreen plants of the genus Coffea. The two most commonly grown species are Coffea robusta (also known as Coffea canephora) and Coffea arabica. These are cultivated in Latin America, Southeast Asia, and Africa. Concentrations on total chlorogenic acids (tCGA) are different in the two species. In general, tCGA concentration is higher in Coffea robusta than in Coffea arabica. Table 2 summarizes the content of FQA, CQA, and tCGA in the two coffee species before roasting.

TABLE 2
Content of FQA, CQA, and tCGA in the Coffea arabica and Coffea
robusta

| | Coffea arabica | | Coffea robusta | |
|---|---|---|---|---|
| | g/kg | | g/kg | |
| Phenolic acids | Santos | Sao Paulo | Ghana | Uganda |
| Feruloylquinic acids | 2.3-3.3 | 0-2.1 | 11.6-12.0 | 5.4-6.8 |
| (FQA) | | | | |
| Caffeoylquinic acids | 60.8-62.6 | 56.2-58.2 | 79.2-84.3 | 77.1-80.9 |
| (CQA) | | | | |
| Total chlorogenic acids | 64.2-64.8 | 56.5-59.1 | 92.6-94.7 | 83.9 86.6 |
| (tCGA) | | | | |

Clifford MN and Wright J, 1976

The torrefaction process has the aim of developing the coffee aroma. The traditional roasting method lasts between 15 and 23 minutes depending on the machinery. The coffee beans are gradually heated while being constantly tossed about. At about 100° C., the beans go yellowish and lose a good proportion of their water by evaporation. Towards 150° C., the beans that have become light brown begin to give off an aroma. Between about 200 and 250° C., the beans become a mahogany brown color. If the torrefaction is continued (230° C.), the bean becomes quite black. This change of color is known as the Stücker reaction. Under the effect of heat, certain constituents disappear; others combine with each other to form complex products. In the first 10 minutes, caramelization of sugars occurs from 160° C.: this is known as Maillard's reaction. At the end of about 10 minutes (200° C.), this reaction causes the first 4 aromas to arise from aroma precursor acids. These aromas are destroyed by possible carbonization. After 10 minutes, the bean will have lost most of its water by evaporation. The sugars and tannins gradually disappear. During the torrefaction process, the total chlorogenic acids are partially destroyed. Table 3 shows the destruction of chlorogenic acids as a consequence of the torrefaction process of the coffee. Therefore, it is desirable to avoid the roasting process in order to preserve a high content of chlorogenic acids in the coffee beans.

TABLE 3
Effects of the torrefaction process over the total chlorogenic acids
(tCGA) in the green coffee.

| | | Torrefaction (205° C.) | | | |
|---|---|---|---|---|---|
| | Total | | | | Really |
| Green | chlorogenic | Soft | | Strong | strong |
| coffee beans | acids (tCGA) | (7 min) | Mild | (13 min) | (19 min) |
| Coffea Arabica | 57.6 | 23.8 | 19.8 | 7.1 | 2.2 |
| (Guatemala) | | | | | |
| Coffea robusta | 68.2 | 30.2 | 17.8 | 5.2 | 1.4 |
| (Uganda) | | | | | |

Trugo and Macrae, 1984

Studies managed by the NAT'Life Division of Naturex and INRA (National Institute of Agronomic Research) allowed to clarify chlorogenic acids absorption thanks to in situ stomach infusion, intestinal perfusion models and nutritional intervention experiment in rats. Different absorption sites and different metabolites were identified. The results showed that about 30% of chlorogenic acids are absorbed from the stomach and the small intestine, the other part reaching the colon (FIG. 1). From the stomach, the absorption doesn't induce modifications in the CGA structures. The absorption in the stomach represents about 16%. From the small intestine and the colon, most of chlorogenic acids ingested are hydrolysed in caffeic and quinic acids. In the small intestine, chlorogenic acids are hydrolysed by enterocytes. The caffeic acid liberated is next O-methylated. After that, Caffeic and (iso)ferulic acids go to the blood and could be metabolized into the tissues. In the colon, chlorogenic acids are hydrolyzed by the microflora. Quinic and caffeic acids thus released are: directly absorbed and metabolized into enterocites; metabolized by the intestinal flora, absorbed and metabolized again into colonocytes; and finally excreted in the feces.

It has been demonstrated that tCGA regulates glycemia by inhibiting glucose-6-phosphatase (Glc-6-Pase) activity. Glc-6-Pase plays an important role in the homeostatic control of blood sugar concentration. This enzyme system, only present in the liver, is in fact responsible for the conversion of glucose-6-phosphate into glucose capable of passing into the general circulation. Inhibition of hepatic Glc-6-Pase causes a reduction in the hepatic production of glucose and consequently decreases abnormally high levels of glucose in the blood.

Recent discoveries have shown that 5-caffeoylquinic acid (5-CQA) inhibits the activity of Glc-6-Pase in a specific way, in particular the activity of its Glc-6-Pase translocase 1 unit (T1) (McCarty. Med. Hypotheses. 2001 March; 56(3): 286-289). In vitro and in vivo studies carried out with 5-CQA, the main polyphenol in coffee, showed that this phenolic acid is able to modulate glucose metabolism (Welsch et al. J. Nutr., 1989. 119(11):1698-1704.—Arion et al. Arch Biochem Biophys, 1997. 339(2):315-22.—Herling et al. Am J Physiol, 1998. 274(6 Pt 1): p. G1087-93.—Hsu et al. Planta Med. 2000. 66(3): p. 228-30.—Andrade-Cetto et al. J Ethnopharmacol, 2001. 78(2-3): p. 145-9.—Rodriguez de Sotillo et al. J Nutr Biochem, 2002. 13(12): p. 717-726.—Johnston et al., Am J Clin Nutr, 2003. 78(4). p. 728-33). More particularly, it was shown that 5-CQA inhibits Glc-6-Pase in intact rat microsomes while no effect was shown in fully disrupted microsomes. However, there is no evidence on the inhibition of Glc-6-Pase by other CGA neither by green coffee extract.

Blum et al. (Nutrafoods 2007; 6(3):13-17.) conducted a study in order to determine the hypoglycaemic effect of a green coffee extract (Svetol® green coffee extract, NATUREX) in humans. The aim of the clinical trial was to determine if the green coffee extract could decrease glycemia in the postprandial state in humans. Fifteen healthy women (18-70 y) participated in the study. All participants were used as their own control and were submitted to an oral glucose tolerance test before and after supplementation of the green coffee extract. The supplementation consisted of 600 mg of green coffee extract daily during forty days, divided in three doses of 200 mg each before the meals. Results indicated a significant decrease (147±9.3 vs 133±8.7 mg/dL; p<0.05) in post-load glycemia compared to the one obtained before supplementation (FIG. 2). Moreover at the end of the study, a weight loss of around 1.5 kg was noted. In conclusion, these preliminary results suggest that green coffee extract is able to modulate glucose metabolism and that this modulation could have an effect on weight management.

In another clinical trial, Deallalibera et al. (Phytothérapie expérimentale 2006 November; 4(4): 194-197) studied the effect of a green coffee extract (Svetol® green coffee extract, NATUREX) on body weight loss in humans. Fifty overweight volunteers (BMI>25 kg/m²) were randomized in two groups, control group (n=20) receiving placebo, and treated group (n=30) receiving the green coffee extract (Svetol® green coffee extract, NATUREX) with bland low calorie diet. Each volunteer took one capsule of the 200 mg of green coffee extract twice a day with the main meal, for 60 days. Changes in Muscle Mass/Fat Mass ratio (MM/FM), body weight, body mass index (BMI), and self evaluation of physical aspects were recorded at baseline and at the end of the study. After 60 days, the MM/FM ratio was increased statistically in the green coffee group compared to the placebo: 4.1±0.7% vs. 1.6±0.6% respectively (P<0.01). Moreover, a significant reduction in weight of 4.97±0.32 kg (5.7%), as well as in the BMI (−1.7 kg/m²), were observed in the green coffee extract compared to the placebo (P<0.001). The significant increase of MM/FM ratio and decrease of weight and BMI showed that the green coffee extract (Svetol® green coffee extract, NATUREX) is able to exacerbate effect of a bland low calorie diets on volunteers who are overweight. This effect could be explained by increasing the consumption of fatty deposits and by preventing them from being accumulated.

### SUMMARY OF THE INVENTION

Broadly, the present invention is directed to methods of using green coffee extract, such as Svetol® brand green coffee extract (commercially available from NATUREX S.A., Avignon France), to inhibit Glc-6-Pase, reduce glycemia and promote reduced body fat, increase Muscle Mass/Fat Mass ratio (MM/FM) in the body, and reduce the body weight and body mass index (BMI) in humans. One aspect of the invention is directed to a method of improving body weight in humans and reducing the risk of Type 2 diabetes (T2D) by administering an amount of green coffee extract effective to treat a subject. A preferred green coffee extract contains a ratio of 5-caffeoylquinic acid (5-CQA) and total chlorogenic acids (tCGA) (5-CQA/tCGA) of between about 0.2 and 0.3. More preferably, the concentration of tCGA is greater than about 45% and the concentration of 5-CQA is less than about 10%. A preferred method of administration consists of administering between about 200 mg and about 1,000 mg per day, more preferably administering about 400 mg per day.

The green coffee extract from Coffea robusta beans is preferably obtained by hydroalcoholic extraction with a plant/extract ratio of between 6:1 and 8:1. The alcohol solvent used is exclusively food quality alcohol. Preferably, the green coffee extract, although rich in tCGA and 5-CQA, contains no caffeine. In addition, the choice of raw material and the extraction conditions provide an extract containing no cafestol or kahweol, constituents of coffee which can increase the risk of cardiovascular disease.

The inventors believe they are the first to have discovered one can use a green coffee extract for the inhibition of Glc-6-Pase. The inhibition of Glc-6-Pase is favored by the ratio existing between 5-CQA and tCGA in the green coffee extract. For the first time, it is demonstrated that not only 5-CQA inhibits Glc-6-Pase, but also other CGA present in the green coffee extract. This specially designed green coffee extract is more effective inhibiting Glc-6-Pase than the combination of isolated CGA, evidencing a greater level of synergy between all natural CGA present in the extract.

In one embodiment of the invention, the bioavailability of CGA in humans is defined from the specially designed green coffee extract of the present invention. Data on the bioavailability of CGA from green coffee in humans is nonexistent. The special composition in the green coffee extract allows that three CQA, three diCQA and caffeic, ferulic, isoferulic and p-coumaric acids are bioavailable in plasma; and 4-CQA, 5-CQA and sinapic, p-hydroxybenzoic, gallic, vanillic, dihydrocaffeic, caffeic, ferulic, isoferulic, and p-coumaric acids in urine. Therefore, the CGA in the green coffee extract, which is also able to inhibit the Glc-6-Pase activity, is bioavailable in humans as evidenced in plasma and urine.

In another embodiment of the invention, a green coffee extract (Svetol® green coffee extract, NATUREX) is applied and able to reduce glycemia in humans.

In another embodiment of the invention, a green coffee extract (Svetol® green coffee extract, NATUREX) is applied and able to reduce body fat in humans.

In another embodiment of the invention, a green coffee extract (Svetol® green coffee extract, NATUREX) is applied and able to increase the Muscle Mass/Fat Mass ratio (MM/FM) in humans.

In another embodiment of the invention, a green coffee extract (Svetol® green coffee extract, NATUREX) is applied and able to reduce body weight in humans.

In a further embodiment of the invention, a green coffee extract (Svetol® green coffee extract, NATUREX) is applied and able to reduce Body Mass Index (BMI) in humans.

### BRIEF DESCRIPTION OF THE FIGURES

Further features, advantages and characteristics of the present invention will become apparent to a person of ordinary skill in the art in view of the following detailed discussion of preferred embodiments of the present invention, made with reference to the drawings annexed, in which:

FIG. 1 provides a diagram summarizing chlorogenic acids absorption;

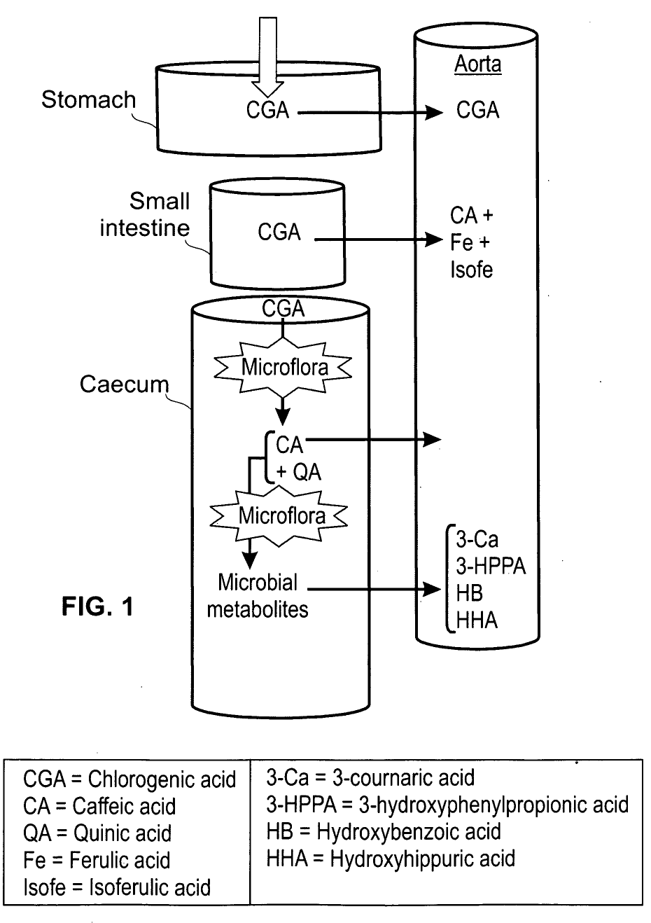

FIG. 2 illustrates an oral glucose tolerance test with and without green coffee extract (Svetol® green coffee extract, NATUREX) supplementation;

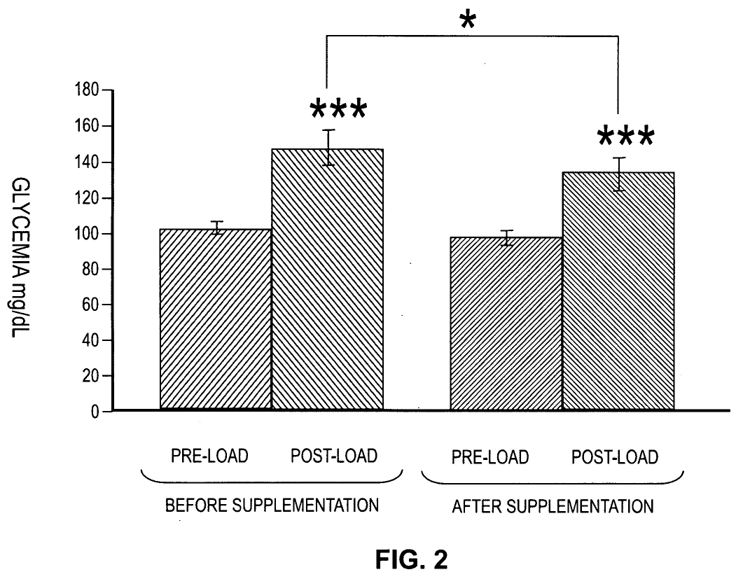

FIG. 3 illustrates the variation (%) of the Muscle Mass/Fat Mass ratio (MM/FM) after 60 days of green coffee extract (Svetol® green coffee extract, NATUREX) supplementation. **P<0.01;

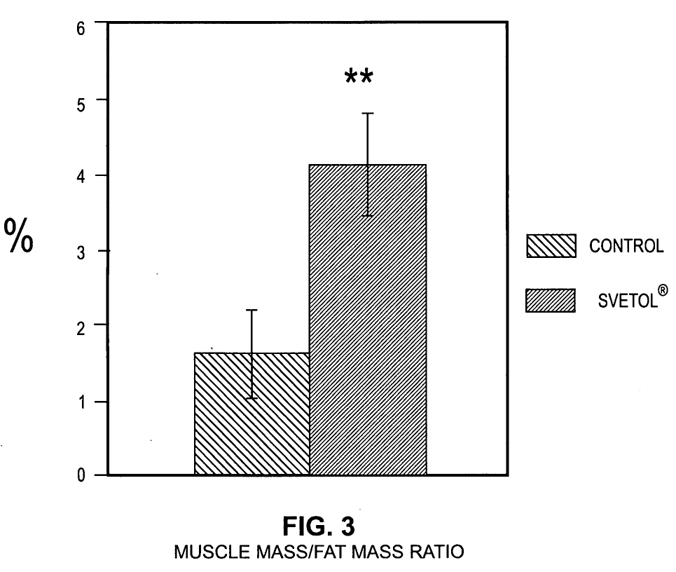

FIG. 4. Weight loss reduction after 60 days of green coffee extract (Svetol® green coffee extract, NATUREX) supplementation. **P<0.001;

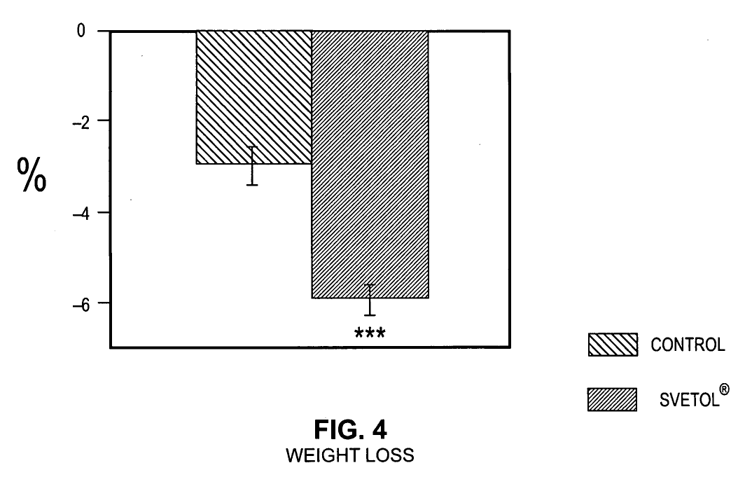

FIG. 5 illustrates Body Mass Index (BMI) reduction after 60 days of green coffee extract (Svetol® green coffee extract, NATUREX) supplementation. **P<0.001;

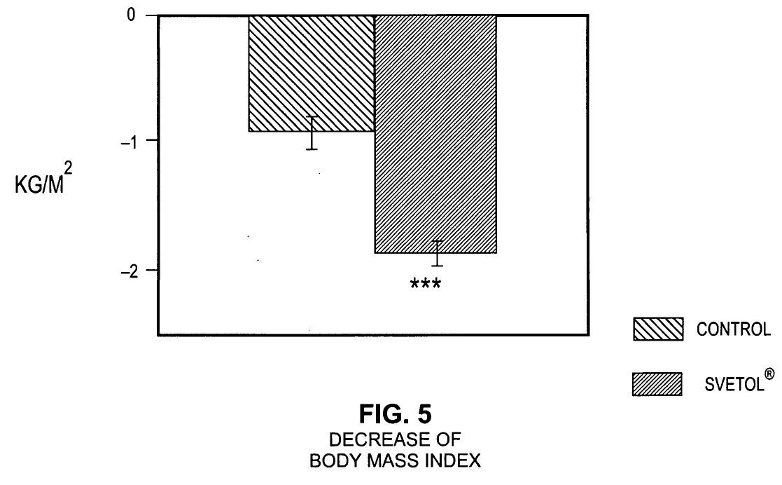

FIG. 6 provides the high-pressure liquid chromatography chemical fingerprint for a green coffee extract (Svetol® green coffee extract, NATUREX);

FIG. 7 illustrates the activity of glucose-6-phosphatase in disrupted human microsomes with or without green coffee extract (Svetol® green coffee extract, NATUREX). Values are mean+/−SD. *p<0.05; **p<0.01; ***p<0.001 vs control;

FIG. 8 illustrates the activity of glucose-6-phosphatase in disrupted human microsomes with or without 3-; 4-caffeoylquinic acids tested alone. Values are mean+/−SD. *p<0.05; **p<0.01; ***p<0.001 vs control;

FIG. 9 illustrates the activity of glucose-6-phosphatase in disrupted human microsomes with or without 5-caffeoylquinic acid (5-CQA) tested alone. Values are mean+/−SD. *p<0.05; **p<0.01; ***p<0.001 vs control;

FIG. 10 illustrates the activity of glucose-6-phosphatase in disrupted human microsomes with or without 3-; 4-; 5-caffeoylquinic mixed together. Values are mean+/−SD. *p<0.05; **p<0.01; ***p<0.001 vs control;

FIG. 11 illustrates the activity of glucose-6-phosphatase in intact human microsomes with or without green coffee extract (Svetol® green coffee extract, NATUREX). Values are mean+/−SD. *p<0.05; **p<0.01; ***p<0.001 vs control;

FIG. 12 illustrates the activity of glucose-6-phosphatase in intact human microsomes with or without 3-caffeoylquinic acids tested alone. Values are mean+/−SD. *p<0.05; **p<0.01; ***p<0.001 vs control;

FIG. 13 illustrates the activity of glucose-6-phosphatase in intact human microsomes with or without 4-caffeoylquinic acids tested alone. Values are mean+/−SD. *p<0.05; **p<0.01; ***p<0.001 vs control;

FIG. 14 illustrates the activity of glucose-6-phosphatase in intact human microsomes with or without 5-caffeoylquinic acid (5-CQA) tested alone. Values are mean+/−SD. *p<0.05; **p<0.01; ***p<0.001 vs control; and

FIG. 15 illustrates the activity of glucose-6-phosphatase in intact human microsomes with or without 3-; 4-; 5-caffeoylquinic mixed together. Values are mean+/−SD. *p<0.05; **p<0.01; ***p<0.001 vs control.

### DESCRIPTION OF THE INVENTION

With reference to the drawings, the present invention related to methods of using green coffee extract, such as Svetol® green coffee extract (NATUREX), to, among other things, inhibit Glc-6-Pase, reduce glycemia, reduce body fat, increase the Muscle Mass/Fat Mass ratio (MM/FM) in the body, and reduce the body weight and body mass index (BMI) in humans, as will now be described through the following examples. As noted, in a preferred embodiment, the green coffee extract from Coffea robusta beans is obtained by hydroalcoholic extraction with a plant/extract ratio of between 6:1 and 8:1. The alcohol solvent used is exclusively food quality alcohol. This preferred green coffee extract, although rich in tCGA and 5-CQA, contains no caffeine, cafestol or kahweol.

### EXAMPLES

### Example 1

### Nutritional Profiles of a Green Coffee Extract (Svetol® Green Coffee Extract, NATUREX)

Nutritional analyses were conducted according to the European pharmacopoeia. Table 4 shows the nutritional value of the green coffee extract used in the examples reported herein (Svetol® green coffee extract, NATUREX).

TABLE 4
Nutritional profile of the green coffee extract (Svetol ® green coffee
extract, NATUREX).

| Compound | Concentration |
|---|---|
| Loss of drying [JO 03/11/1977] | 5% |
| Proteins (Nx6.25) [JO 03/11/1977] | 9% |
| Lipids [JO 03/11/1977] | 1% |
| Ash [JO 03/11/1977] | 10% |
| Soluble sugars [JO 03/11/1977] | 24% |
| Caffeine [HPLC] | 1% |
| Total polyphenols [Folin-Ciocalteau] | 50% |
| (tCGA) Total chlorogenic acids [HPLC] | 45% |
| (5 CQA) 5-caffeoylquinic acid [HPLC] | >10% |
| Cafestol [HPLC] | <2 ppm |
| Kahweol [HPLC] | <2 ppm |
| TOTAL | 100% |

The ratio between 5-caffeoylquinic acid (5-CQA) and total chlorogenic acids (tCGA) (5-CQA/tCGA in Table 4) of the green coffee extract (Svetol® green coffee extract, NATUREX) is between 0.2 and 0.3.

### Example 2

### HPLC Chromatogram of the Green Coffee Extract (Svetol® Green Coffee Extract, NATUREX)

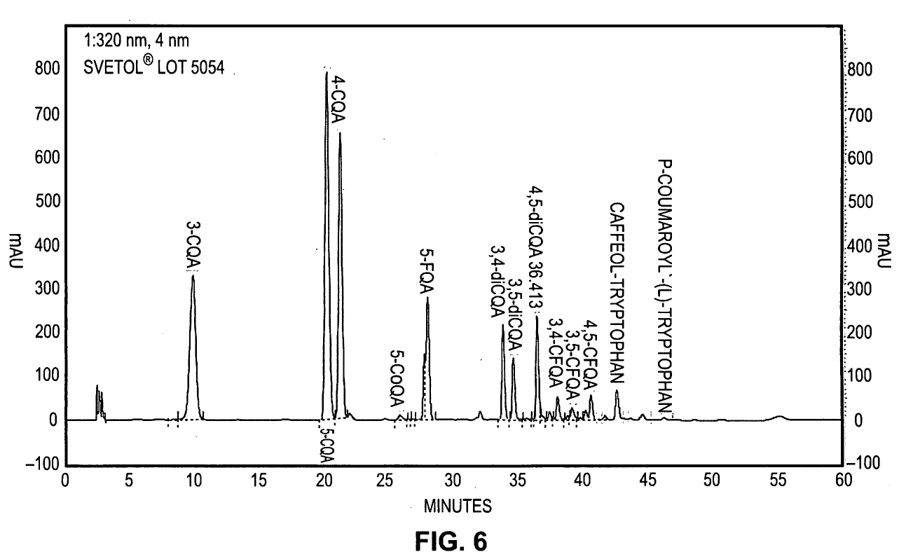

The high-pressure liquid chromatography chemical fingerprint for a green coffee extract (Svetol® green coffee extract, NATUREX) is presented in FIG. 6. The method for performing this analysis was as follows: HPLC-DAD was achieved using a column RP-C₁₈ (5 μm-250×4.6 mm) at 55° C. The flow rate was 0.8 mL/min, and the elution was monitored at 330 nm. The mobile phases were (A) distilled water+H₃PO₄ 0.002M, and (B) acetonitrile. A solution of 100% A was maintained during 8 min, increased by linear gradient to 35% A and 65% B by volume after 35 min total time; followed by a linear gradient of 100% A after maintaining this composition for 10 min; the system was then re-equilibrated to the initial composition. The content of CGA from 10 batches of green coffee extract (Svetol® green coffee extract, NATUREX) reported as mean±standard deviation (SD) is shown in Table 5.

TABLE 5
Chlorogenic acids (CGA) of the green coffee extract (Svetol ® green
coffee extract, NATUREX).

| | | Standard |
|---|---|---|
| Compound | Concentration % | Deviation |
| 3-caffeoylquinic acid (3-CQA) | 8.91 | 0.71 |
| 4-caffeoylquinic acid (4-CQA) | 10.47 | 0.74 |
| 5-caffeoylquinic acid (5-CQA) | 12.55 | 0.84 |
| 4-feruloylquinic acid (4-FQA) | 0.00 | 0.00 |
| 5-feruloylquinic acid (5-FQA) | 4.47 | 0.18 |
| 3,4-dicaffeoylquinic acid (3,4-diCQA) | 2.63 | 0.15 |
| 3,5-dicaffeoylquinic acid (3,5-diCQA) | 1.72 | 0.14 |
| 4,5-dicaffeoylquinic acid (4,5-diCQA) | 2.90 | 0.22 |
| 3,4-caffeoylferuloylquinic acid (3,4- | 0.62 | 0.02 |
| CFQA) | | |
| 3,5-caffeoylferuloylquinic acid (3,5- | 0.18 | 0.16 |
| CFQA) | | |
| 4,5-caffeoylferuloylquinic acid (4,5- | 0.66 | 0.03 |
| CFQA) | | |
| 5-Coumaroylquinic acid (5-CoQA) | 0.18 | 0.02 |
| Caffeic acid | 0.00 | 0.00 |
| Caffeoyl-tryptophan | 0.99 | 0.09 |
| p-coumaroyl-tryptophan | 0.13 | 0.04 |
| Total | 46.41 | 1.35 |

### Example 3

### Composition of Total Chlorogenic Acids (tCGA) and 5-caffeoylquinic Acid (5-CQA) in Several Coffee Extracts

Table 6 shows the composition of chlorogenic acids (tCGA) and 5-caffeoylquinic acid (5-CQA), as well as the ratio 5-CQA/tCGA, of several commercial extracts. All samples were analyzed using the HPLC method described in Example 2. The last two rows include the values of the green coffee extract described in this invention (Svetol® green coffee extract, NATUREX).

TABLE 6
Composition of total chlorogenic acids (tCGA) and 5-caffeoylquinic
acid (5-CQA) in several coffee extracts

| | | | Ratio 5-CQA/ | |
|---|---|---|---|---|
| Coffee extract | tCGA (HPLC) | 5-CQA | tCGA | Specie |
| Svetol ®, Naturex¹ | 46.41 | 12.55 | 0.27 | C. robusta |
| Commercial sample 1 | 5.57 | 2.2 | 0.39 | C. arabica |
| Commercial sample 2 | 6.07 | 2.2 | 0.36 | C. arabica |
| Commercial sample 3 | 5.55 | 1.98 | 0.36 | C. arabica |
| Commercial sample 4 | 50 | 24 | 0.48 | C. arabica |
| Commercial sample 5 | 35 | 18 | 0.51 | C. arabica |
| Commercial sample 6 | 45 | 19 | 0.42 | C. arabica |
| Commercial sample 7 | 27 | 5 | 0.19 | C. arabica |
| Commercial sample 8 (Batch 1) | 53 | / | | C. arabica |
| Commercial sample 8 (Batch 2) | 45 | 19 | 0.42 | C. arabica |
| Commercial sample 9 (Batch 1) | 79 | 50 | 0.63 | C. arabica |
| Commercial sample 10 | 78 | 59 | 0.76 | C. arabica |
| Commercial sample 11 | 64 | 30 | 0.47 | C. arabica |
| Commercial sample 13 | 79 | 51 | 0.65 | C. robusta |
| Commercial sample 14 | 70.2 | 61.5 | 0.88 | C. robusta |
| Commercial sample 15 | 48 | 21 | 0.44 | C. arabica |
| Commercial sample 16 | 43 | 16 | 0.37 | C. robusta |
| Commercial sample 17 | 66 | | | |

¹Mean of 10 batches

### Example 4

### Inhibition of Hepatic Glucose-6-phosphatase (Glc-6-Pase) by Green Coffee Extract (Svetol® Green Coffee Extract, NATUREX)

The aim was to determine if a decaffeinated green coffee extract (Svetol® green coffee extract, NATUREX) is able to inhibit the glucose-6-phophatase (Glc-6-Pase) system and to determine which type of chlorogenic acids, 3-; 4- or 5-caffeoylquinic acids, is the best active molecule.

Glc-6-Pase activity was assayed by quantifying orthophosphate formation, as described previously (Arion et al. Arch Biochem Biophys 1997; 339(2):315-22). The enzyme assays were conducted in a final rectional volume of 320 μL, containing different concentrations of glucose-6-phosphate ranging from 1 to 20 mM, 100 mM cacodylic acid pH 6.5. The reaction was initiated by the addition of intact or disrupted microsomes, for 5 minutes and was stopped by the addition of 3.2 mL of calorimetric reagent (six volumes of acid molybdate (0.42% ammonium molybdate in 1 N H₂SO₄), two volumes of 5% SDS and one volume of 10% ascorbic acid, freshly prepared and stored in ice for maximum 6 hours). All samples were then incubated for 30 minutes at 45° C., and the amount of phosphate liberated per minute was determined as the blue phosphomolybdous complex at 820 nm.

Experiments were conducted with or without addition of the green coffee extract (Svetol® green coffee extract, NATUREX) (final concentration of total chlorogenic acids: 0.4 or 0.6 mM) but also with isolated caffeoylquinic acids. The final concentration of pure molecules tested (3-, 4- and 5-caffeoylquinic acids alone or mixed) corresponded to their concentration in green coffee extract tested: 0.08, 0.08, 0.11 and 0.27 mM respectively for the green coffee extract with total chlorogenic acids at 0.4 mM; 0.12, 0.12, 0.17 and 0.41 mM for the green coffee extract with total chlorogenic acids at 0.6 mM. Enzyme activity is expressed as milliunits per milligram of protein. Experiments were conducted in triplicates, and regression analyses of plotted data were carried out using XLStat (Version 2008.1.03, Addinsoft). Comparisons of Glc-6-Pase activities in the presence and absence of the green coffee extract (Svetol® green coffee extract, NATUREX) were performed by one way analysis of variance (ANOVA). The levels of significance was set up at p<0.05.

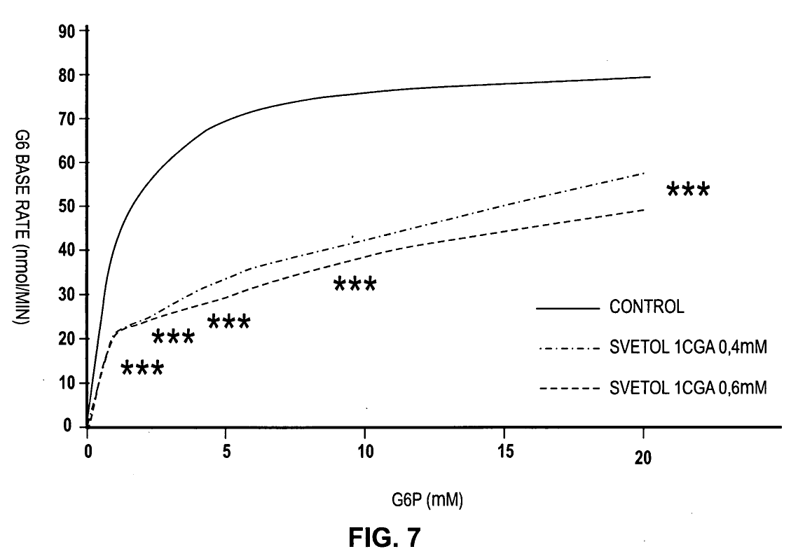

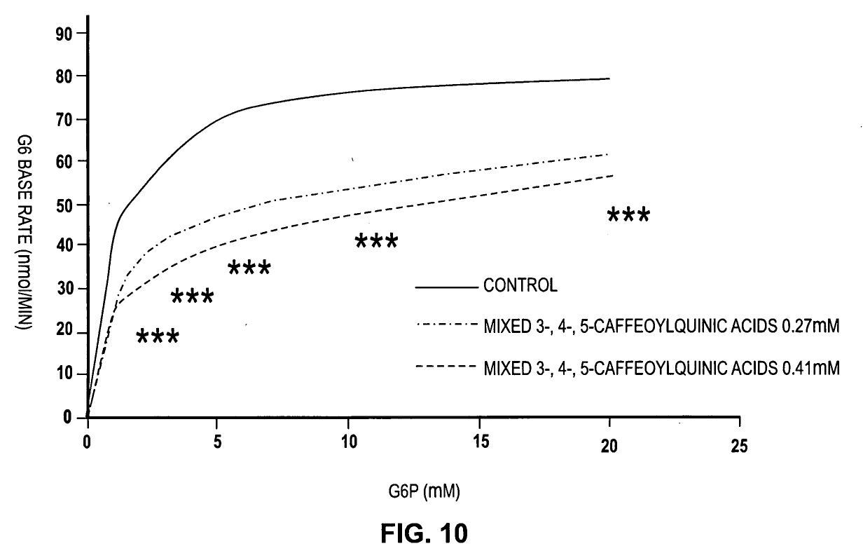

Results of the experiments in disrupted microsomes are shown in FIGS. 7 to 10.

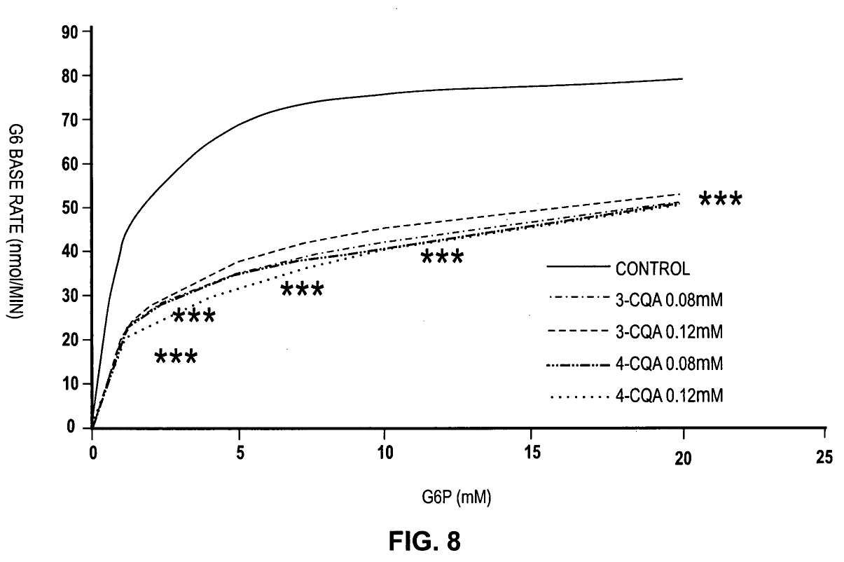

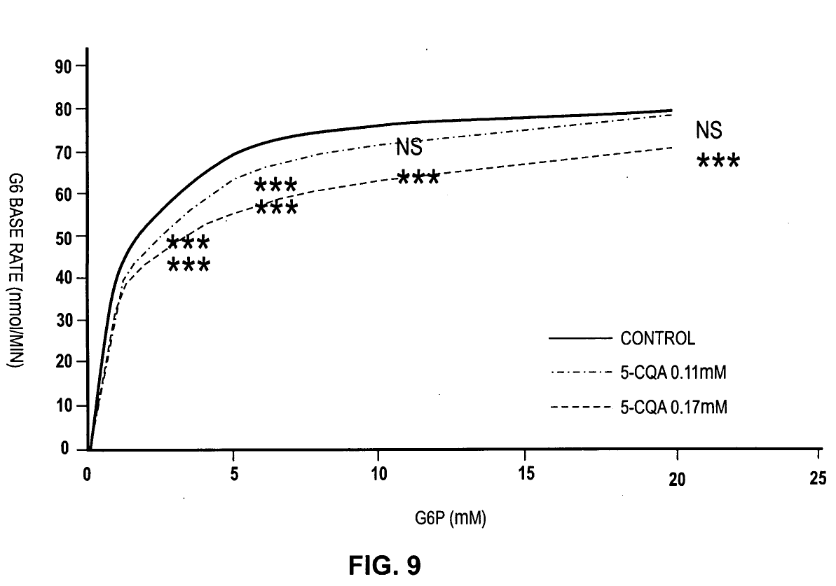

Disrupted microsomes permit to have access directly to the catalytic site. Whatever the concentration of green coffee extract (Svetol® green coffee extract, NATUREX) tested, it inhibits significantly the catalytic site but not in a dose-dependent manner (p<0.001 vs control; FIG. 7). When purified chlorogenic acids were tested separately, all of them inhibit significantly the enzyme, however, activities of 3- and 4-caffeoylquinic acids are higher than those of 5-caffeoylqunic acid (FIGS. 8 and 9). When purified chlorogenic acids are mixed together, no synergic effect was shown, the inhibition effect results from the sum of each chlorogenic acid effect independently (p<0.001). No dose-dependent effect was shown like the green coffee extract (Svetol® green coffee extract, NATUREX). Moreover the sum of these activities explained a part of the green coffee extract (Svetol® green coffee extract, NATUREX) effect but not the totality suggesting that the dicaffeoylquinic acids present in the green coffee extract (Svetol® green coffee extract, NATUREX) are probably also active towards the Glc-6-Pase catalytic site.

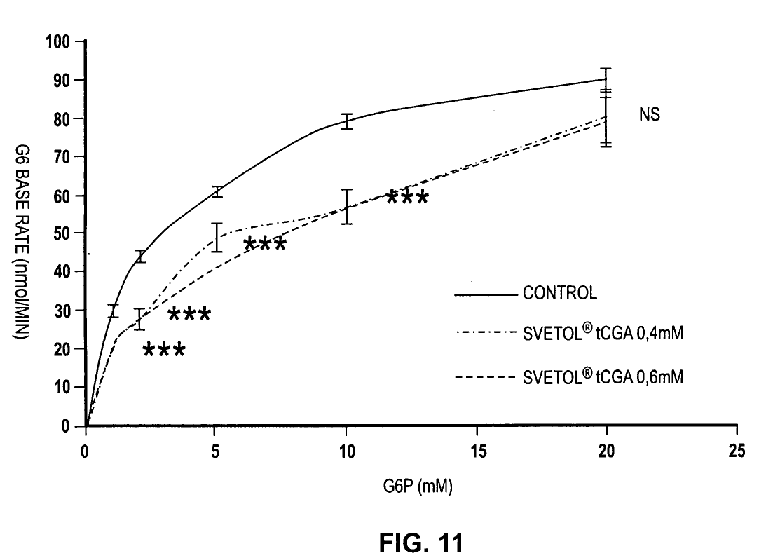

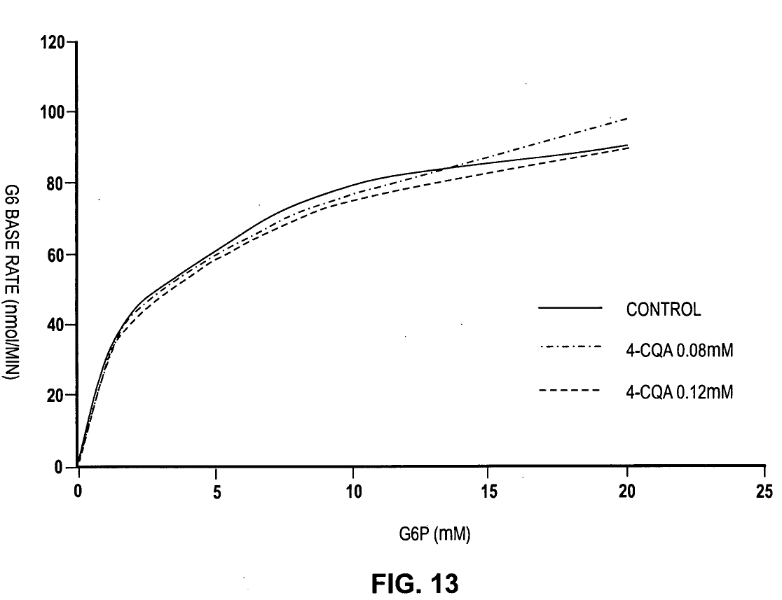

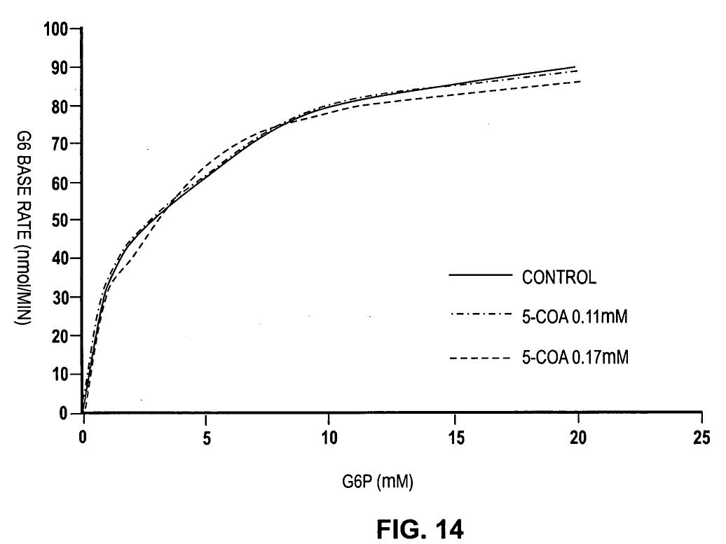

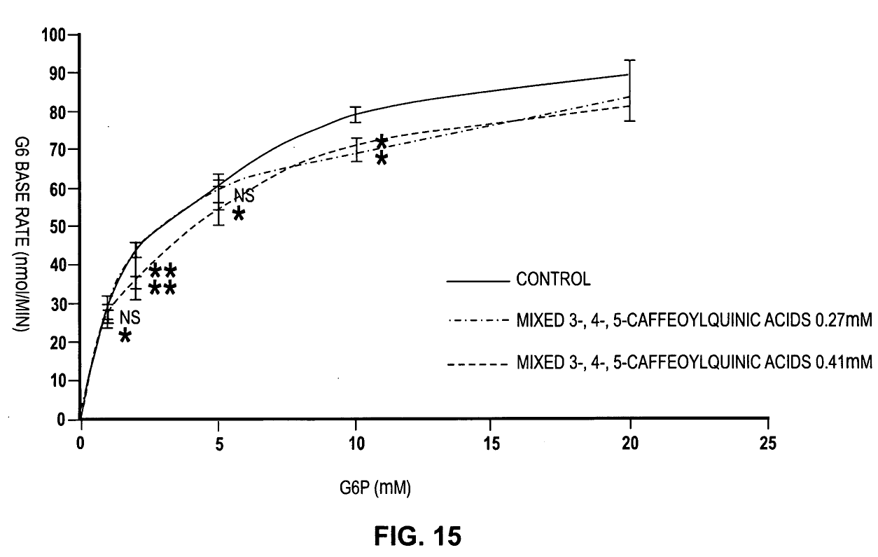

Results of the experiments in intact microsomes are shown in FIGS. 11 to 15.

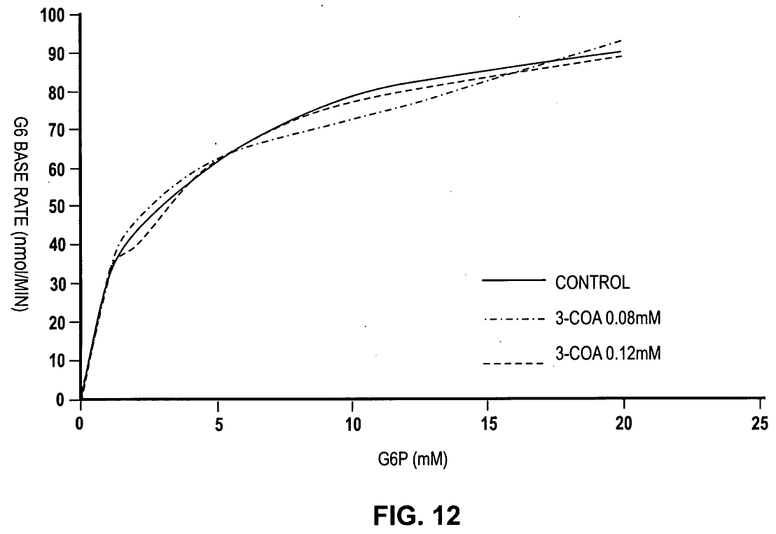

Intact microsomes permit detection of the effect of the green coffee extract (Svetol®) green coffee extract, NATUREX) and chlorogenic acids towards a Glc-6-Pase translocase, denoted T1, which facilitates penetration of glucose-6-phosphate into the endoplasmic reticulum. Such as for disrupted microsomes and whatever the concentration of the green coffee extract (Svetol® green coffee extract, NATUREX) tested, it inhibits significantly the translocase but not in a dose-dependent manner (p<0.001 vs control; FIG. 11). Surprisingly, when purified chlorogenic acids were tested separately, none of them have an activity towards the transporter T1 (FIGS. 12, 13, 14 and 15). When chlorogenic acids are mixed, the inhibition is significant compared to the control (p<0.05 or p<0.01 vs control). Such as for disrupted microsomes the sum of these activities explained a part of the green coffee extract (Svetol® green coffee extract, NATUREX) effect but not the totality suggesting that the dicaffeoylquinic acids present in the green coffee extract (Svetol® green coffee extract, NATUREX) are probably also active towards the translocase.

At the conclusion of this study, it appeared that the green coffee extract (Svetol® green coffee extract, NATUREX) is able to inhibit the catalytic site and the translocase of the Glc-6-Pase. 3-, 4- and 5-caffeoylquinic acids present in the green coffee extract (Svetol® green coffee extract, NATUREX) explains a part of the extract's activity but not the totality, suggesting that the dicaffeoylquinic acids present in the green coffee extract also probably have an activity towards this enzymatic system.

Until now, only 5-caffeoylquinic acid (5-CQA) was tested. The present invention shows for the first time that 3- and 4-caffeoylquinic acids are more active than 5-caffeoylquinic acid. Moreover, the major part of the green coffee extract (Svetol® green coffee extract, NATUREX) activity being explained by the sum of the pure molecule activity, the standardization of the extract in these molecules but also in total chlorogenic acids (including dicaffeoylquinic acids) is very important to develop an extract with health reproducible effect.

### Example 5

### Bioavailability of Total Chlorogenic Acids of a Green Coffee Extract (Svetol® Green Coffee Extract, NATUREX) in Humans

The objective of this study was to evaluate the pharmacokinetic profiles of CGA compounds and metabolites in human plasma and urine after the acute consumption of a decaffeinated green coffee extract and to estimate the apparent bioavailability of CGA in this food matrix.

A preliminary test on 2 volunteers administrated with 400 mg and 1,000 mg of a green coffee extract (Svetol® green coffee extract, NATUREX) showed that doses up to 400 mg did not increase the concentration of total caffeoylquinic acids (tCQA), total dicaffeoylquinic acids (tdiCQA), and total chlorogenic acids (tCGA) in plasma (Table 7). These result evidence saturation at concentrations up to 400 mg in humans. Therefore, 400 mg can be considered a suitable dose of green coffee extract (Svetol® green coffee extract, NATUREX) in order to guarantee an appropriate tCGA concentration in human plasma.

TABLE 7
Pharmacokinetic parameters of chlorogenic acids compounds
identified in plasma during 2 h after consumption of 400 mg and
1,000 mg of a green coffee extract (Svetol ® green coffee
extract, NATUREX).

| Dose | tCQA | tdiCQA | tCGA |
|---|---|---|---|
| Volunteer 1 | | | |
| 400 mg | 6.06 | 5.67 | 11.73 |
| 1,000 mg | 5.99 | 2.65 | 8.64 |
| Volunteer 2 | | | |
| 400 mg | 7.41 | 2.50 | 9.91 |
| 1,000 mg | 4.26 | 1.93 | 6.19 |

In a further phase of the study, ten non-smoker volunteers (22-55 y), five male and five female, were recruited. Subjects were instructed to avoid consumption of phenolic-containing foods during the 48 h prior to the study. They were asked to eat only animal foods, refined cereal foods and artificial beverages. On the day of the study, after 10-12 h overnight fasting, an I.V. catheter was inserted into the antecubital vein and a baseline heparinized blood sample was obtained. 400 mg green coffee extract (Svetol® green coffee extract, NATUREX) were offered to each subject and sequential blood draws were obtained 0.5; 1; 2; 3; 4; 5; 6; 7 and 8 h after the capsules consumption. Blood samples were collected into heparin-containing tubes. Baseline blood aliquots were used to determine hematocrit and hemoglobin levels by standard methods. Plasma samples were obtained by centrifugation of the blood samples immediately after being drawn. Urine samples were also collected at baseline interval (minus 2-0 h) and at intervals of 0-2 h; 2-4 h; 4-6 h and 6-8 h after coffee consumption into appropriate plastic containers. Total urine volume was measured for each collection period. Plasma and urine aliquots for determination of CGA were acidified with HCl and kept frozen in liquid nitrogen until analyses. Urine aliquots for determination of creatinine were acidified with HCl and kept at −20° C. until analyses. Every hour, starting one hour after green coffee extract consumption, subjects ate a CGA-free snack composed of white bread (25 g) with cream cheese (15 g) and 100 mL of a saline solution containing 0.21 g of NaCl, 2.28 g of glucose, 0.22 g of potassium citrate monohydrate and 0.1 g of sodium citrate di-hydrate, until the end of blood draws.

Analyses of CGA (including CGA lactones and caffeoyltryptophan) in the green coffee extract (Svetol® green coffee extract, NATUREX), plasma and urine were performed by HPLC and LC-DAD-MS gradient systems as described in detail by Farah et al., (J Agric Food Chem. 2006; 54:374-81) and Monteiro et al (J Nutr.; 137:2196-201). The detection limit for 5-CQA (4-fold baseline noise) under the conditions used in this study was 0.01 μg/mL. Results of CGA and phenolic acids in urine were normalized by creatinine excretion. Molar ratios of specific CGA compounds were calculated in green coffee extract as ratios of total amounts and, in plasma, as ratios of the corresponding AUC.

After green coffee extract (Svetol® green coffee extract, NATUREX) consumption, 3-CQA, 4-CQA, 5-CQA, 3,4-diCQA, 3,5-diCQA and 4,5-diCQA were identified in the plasma of all subjects. Such compounds represented together about 82% of CGA composition of the green coffee extract. Caffeic, ferulic, isoferulic and p-coumaric acids, which were not detected in the encapsulated extract, were present in the plasma of different subjects after green coffee extract consumption, contributing to 6.6%, 6.2%, 6.1% and 1.4% of total phenolics in plasma respectively.

Cₘₐₓ, Tₘₐₓ and AUC of the CGA and cinnamic acids identified in plasma of the 10 subjects after green coffee extract consumption are shown in Table 8. CGA Cₘₐₓ and Tₘₐₓ varied largely among the subjects; Cₘₐₓ of total CQA varied from 0.6 to 16.9 μmol/L, Cₘₐₓ of total diCQA varied from 0.3 to 22.8 μmol/L, whereas Cₘₐₓ of total CGA varied from 1.2 to 39.7 μmol/L, with mean concentrations of 8.2, 6.6 and 14.8 μmol/L, respectively. Tₘₐₓ for total CQA, total diCQA and total CGA varied considerably among the subjects (from 0.5 to 8 h), with mean values of 3.3; 3.2 and 3.1 h, respectively.

Regarding individual compounds, 5-CQA was the major CGA identified in the plasma of all subjects at all time points after green coffee extract consumption, as indicated by both Cₘₐₓ and AUC of 5-CQA. Considering mean values of plasma AUC, 5-CQA, 4-CQA and 3-CQA contributed with 31.3%, 7.5% and 5.2% of AUC of total phenolic compounds in plasma.

Molar ratios among CGA compounds were calculated considering their content in the green coffee extract and the AUC in plasma. For CQA, ratios of 5-CQA:4-CQA:3-CQA in the green coffee extract were 1.2:1.0:1.1, whereas their corresponding ratios in plasma were 6.0:1.4:1.0. The molar ratios 3,5-diCQA:4,5-diCQA:3,4-diCQA in the coffee extract were 1.0:1.6:1.7, while in plasma the ratios were 1.7:1.4:1.0, respectively. Moreover, comparing both CGA classes, the molar ratio diCQA:CQA in plasma was 6.2× higher than in the green coffee extract.

TABLE 8
Pharmacokinetic parameters of chlorogenic acids and
hydroxycinnamic acids identified in plasma after decaffeinated
green coffee consumption.

| | Cmax | | |
|---|---|---|---|
| Compound | (μmol/L) | Tmax (h) | AUC (μmol · h/L) |
| 3-caffeoylquinic acid | 0.9 ± 1.4 | 4.0 ± 2.6 | 3.0 ± 4.5 |
| 4-caffeoylquinic acid | 1.4 ± 1.1 | 3.6 ± 2.2 | 4.3 ± 5.4 |
| 5-caffeoylquinic acid | 5.9 ± 4.2 | 3.3 ± 2.4 | 17.9 ± 15.3 |
| 3,4-dicaffeoylquinic acid | 1.5 ± 1.6 | 2.6 ± 1.8 | 5.0 ± 4.9 |
| 3,5-dicaffeoylquinic acid | 2.7 ± 2.7 | 3.2 ± 2.5 | 8.7 ± 8.3 |
| 4,5-dicaffeoylquinic acid | 2.5 ± 3.0 | 3.3 ± 2.5 | 6.8 ± 5.7 |
| Total caffeoylquinic acids | 8.2 ± 6.3 | 3.3 ± 2.4 | 25.2 ± 24.4 |
| Total dicaffeoylquinic acids | 6.6 ± 6.9 | 3.2 ± 2.5 | 20.4 ± 17.5 |
| Total chlorogenic acids | 14.8 ± 11.7 | 3.1 ± 2.6 | 45.6 ± 37.1 |
| Caffeic acid | 1.1 ± 0.9 | 3.6 ± 2.1 | 3.8 ± 3.2 |
| Ferulic acid | 0.8 ± 0.3 | 2.9 ± 1.8 | 3.6 ± 1.5 |
| Isoferulic acid | 0.9 ± 0.2 | 2.9 ± 1.8 | 3.5 ± 1.9 |
| p-coumaric acid | 0.4 ± 0.03 | 2.5 ± 1.8 | 0.8 ± 0.2 |

Values are Mean ± SD, n = 10.
Cₘₐₓ - Maximum plasma concentration;
Tₘₐₓ - Time corresponding to Cₘₐₓ;
AUC - Area under the curve.

Most subjects presented phenolic compounds in their baseline urine. Trace amounts of 5-CQA were observed in 5 subjects, with contents varying up to 0.35 μmol. Sinapic gallic, p-hydroxybenzoic, and dihydrocaffeic acids were the major phenolic compounds at baseline, representing about 82% of the total amount of the identified phenolic compounds, which varied from 2.6 to 97.0 μmol among the subjects. The urinary excretion of phenolic compounds increased in 9 of 10 subjects after green coffee extract consumption. The total urinary excretion (μmol) of phenolic compounds for each subject before and after green coffee consumption, corrected by creatinine values is shown in Table 9. As with plasma, a large inter-individual variation was observed in the urinary excretion of all compounds after green coffee extract consumption. The only intact CGA compounds identified in urine after the extract consumption were 5-CQA and 4-CQA (0.41 to 4.02 μmol of 5-CQA and 0.83 to 1.22 μmol of 4-CQA). Not only at baseline but also after the extract consumption, sinapic, gallic, p-hydroxybenzoic, and dihydrocaffeic acids were the major phenolic compounds, representing, on average, 85% of the total amount of phenolic compounds identified in urine. Protocatechuic, dihydroferulic, benzoic and hippuric acids, which have been previously identified in urine alter CGA consumption, were not identified in the urine of any of the subjects before or after the extract consumption.

In conclusion, these results increase evidence that at least CQA and diCQA, which are major CGA compounds from coffee absorbed in the human body, are being differentially absorbed and/or metabolized throughout the whole gastrointestinal tract. These results also confirm that urine is not a major excretion pathway of intact CGA compounds and their metabolites, and identifies sinapic, gallic, p-hydroxybenzoic, and dihydrocaffeic acids as major urinary metabolites of CGA in humans. In addition, this study shows that the major CGA compounds present in green coffee matrix are highly bioavailable in humans. A large inter-individual variation clearly exists in CGA absorption and/or metabolism in humans and requires further investigation regarding differences in genetic polymorphisms.

TABLE 9
Total Urinary excretion of CGA and metabolites in each subject after decaffeinated green coffee consumption.

| | | | Dihydro- | | | | | | | | | ρ- | |
|---|---|---|---|---|---|---|---|---|---|---|---|---|---|
| | Gallic | ρ-Hydroxy- | caffeic | Vanillic | Siringic | Sinapic | | 4- | Caffeic | Ferulic | Isoferulic | coumaric | Total |
| | Acid | benzoic acid | acid | acid | acid | acid | 5-CQA | CQA | acid | acid | acid | acid | Phenolics |
| 1-Baseline | 9.24 | 4.94 | 2.98 | 2.61 | 0.04 | 1.26 | Nd | Nd | Nd | 0.45 | 0.01 | 0.01 | 21.54 |
| 0-8 h | 43.47 | 32.58 | 29.97 | 10.38 | 3.85 | 10.50 | Nd | Nd | 0.11 | 2.24 | 0.92 | 0.13 | 134.15 |
| 2-Baseline | 3.55 | 1.08 | 0.25 | 1.86 | 0.47 | 0.97 | Nd | Nd | Nd | 0.13 | 0.02 | 0.01 | 8.34 |
| 0-8 h | 18.67 | 24.14 | 15.81 | 16.37 | 11.07 | 17.52 | 0.42 | Nd | 0.86 | 3.33 | 1.02 | 0.14 | 109.35 |
| 3-Baseline | 1.86 | 2.56 | Nd | 3.31 | 3.52 | 2.22 | 0.02 | Nd | 0.01 | 0.23 | 0.07 | 0.03 | 13.81 |
| 0-8 h | 9.41 | 27.94 | 56.05 | 24.27 | 16.51 | 13.34 | 0.41 | Nd | 0.07 | 4.96 | 2.16 | 0.07 | 155.18 |
| 4-Baseline | 0.70 | 0.28 | 0.16 | 0.41 | 1.62 | 1.43 | Nd | Nd | 0.03 | 0.55 | 0.02 | Nd | 5.22 |
| 0-8 h | 7.98 | 4.69 | 13.60 | 9.47 | 16.63 | 12.08 | 0.60 | Nd | 0.21 | 4.60 | 1.98 | 0.03 | 71.86 |
| 5-Baseline | 10.19 | 1.78 | 5.02 | 9.42 | 13.11 | 20.28 | Nd | Nd | Nd | 1.94 | 0.14 | Nd | 61.89 |
| 0-8 h | 25.45 | 16.65 | 15.09 | 21.20 | 9.35 | 47.67 | 0.46 | Nd | 0.40 | 6.43 | 1.87 | Nd | 144.56 |
| 6-Baseline | 10.57 | 12.03 | 4.25 | 2.73 | 0.34 | 19.55 | 0.04 | 0.04 | 0.09 | Nd | Nd | Nd | 49.63 |
| 0-8 h | 115.87 | 189.74 | 61.33 | 12.94 | 1.52 | 130.47 | 2.33 | 0.90 | 1.41 | 1.76 | 4.68 | 2.84 | 527.23 |
| 7-Baseline | 0.25 | 4.12 | 2.13 | 0.19 | 1.36 | 0.98 | Nd | Nd | Nd | Nd | Nd | Nd | 9.03 |
| 0-8 h | 14.03 | 26.72 | 19.10 | 5.63 | 15.08 | 19.94 | 4.02 | 0.91 | 3.28 | 1.17 | 2.15 | 0.92 | 116.06 |
| 8-Baseline | 6.79 | 12.12 | 27.54 | Nd | 3.64 | 0.87 | 0.35 | Nd | 0.40 | 0.09 | 0.73 | 0.03 | 52.57 |
| 0-8 h | 68.21 | 50.45 | 67.45 | 0.58 | 25.62 | 15.06 | 1.22 | 0.83 | 3.60 | 2.39 | 11.80 | 0.10 | 249.35 |
| 9-Baseline | 1.68 | 1.49 | 3.00 | 3.08 | 1.58 | 5.32 | 0.01 | Nd | 0.11 | 0.03 | 0.51 | Nd | 14.31 |
| 0-8 h | 57.69 | 48.45 | 97.73 | 2.26 | 7.94 | 161.54 | 1.67 | 0.86 | 5.35 | 5.80 | 17.00 | 1.81 | 408.92 |
| 10-Baseline | 25.25 | 19.91 | 12.71 | 0.86 | Nd | 39.11 | 0.21 | Nd | 1.72 | 0.21 | 4.83 | 0.05 | 104.86 |
| 0-8 h | 112.22 | 146.38 | 73.92 | 3.20 | Nd | 178.79 | 1.75 | 1.22 | 9.43 | 3.83 | 18.78 | 9.76 | 559.29 |

Results (μmol) are expressed as total excretion before and during 8 h after decaffeinated green coffee consumption;
Nd = not detected.

### Example 6

### Hypoglycemic Effect of Coffee Extracts in Humans

The aim of this study was to evaluate the acute hypoglycemic effect of coffee extracts (Thom. J Int Med Res 2007; 35:900-908). The study was designed as a three way double-blind randomized crossover study with each subject serving as his or her own control. The products tested are shown in Table 10. 12 volunteers were recruited (BMI<25 kg/m²). After overnight fasting an oral glucose tolerance test (placebo) was performed on all volunteers. Glucose levels were followed for 2 h after intake with measurements at 15, 30, 45, 60, 90 and 120 min and they were immediately randomized to one of the treatments, with glucose levels again followed for 2 h after intake with measurements at 15, 30, 45, 60, 90 and 120. There was a 1 week washout period between the different treatments.

TABLE 10
Coffee products tested

| Sample | Species | Ingredients | Dose |
|---|---|---|---|
| Control | | | 25 g sucrose + 400 ml |
| | | | water |
| Product A | C. arabica + | 91% | 10 g Product A + 25 g |
| | C. robusta | | sucrose + 400 ml water |
| | C. robusta | 9% | |
| | (Svetol ® green | | |
| | coffee extract, | | |
| | NATUREX) | | |
| Product B | | | 10 g Product B + 25 g |
| | | | sucrose + 400 ml water |
| Product C | | | 10 g Product C + 25 g |
| | | | sucrose + 400 ml water |

The results for the glycemic-AUC are shown in Table 11. 10 g of the Product A, Coffee Slender®, containing 900 mg of Svetol® green coffee extract (NATUREX) significantly reduced in 6.9% the glycemic-AUC compared to the placebo group (P<0.05).

TABLE 11
Mean ± SE area under the curve (AUC) data for plasma glucose
concentration over 120 min study period following intake of samples
and a glucose challenge.

| | Control | Product A | Product B | Product C |
|---|---|---|---|---|
| Plasma | 778 ± 10.2 | 724 ± 8.2* | 788 ± 10.1 | 818 ± 10.9 |
| glucose AUC | | | | |

*P < 0.05

### Example 7

### Body Weight and Body Fat Loss Effects of Coffee Extracts in Humans

The aim of this study was to evaluate the body weight and body fat loss effects of coffee extracts (Thom. J Int Med Res 2007; 35:900-908). The study was designed as a double-blind randomized study. The products tested are shown in Table 12. 30 volunteers were recruited (BMI 27.5-32.0 kg/m²). 15 received the Product A, and 15 received the Product B during 12 weeks.

TABLE 12
Coffee products tested

| Sample | Species | Ingredients | Dose |
|---|---|---|---|
| Product A | C. arabica + | 91% | 11 g Product A, in five |
| | C. robusta | | cups per day |
| | C. robusta | 9% | |
| | (Svetol ® green | | |
| | coffee extract, | | |
| | NATUREX) | | |
| Product B | | | 11 g Product B, in five |
| | | | cups per day |

The results for the body weight loss are shown in Table 13 and the results for the body fat are shown in Table 14. 11 g of the Product A, Coffee Slender®, containing 1,000 mg of Svetol® green coffee extract (NATUREX) significantly reduced in 5.4 kg the body weight and in 3.6% the body fat compared to the placebo group (P<0.05) after 12 weeks of administration.

TABLE 13
Mean ± SE weight for overweight volunteers taking
2 kind of coffee extracts.

| | Weight (kg) | | | | |
|---|---|---|---|---|---|
| | | | | Start − | |
| | Start | Week 4 | Week 12 | Week 12 | P-value |
| Product A | 82.2 ± 4.5 | 83.6 ± 4.1 | 79.8 ± 3.9 | 5.4 ± 0.6 | P < 0.05 |
| Product B | 84.3 ± 4.3 | 83.7 ± 4.1 | 81.6 ± 4.2 | 1.7 ± 0.9 | NS |

TABLE 14
Mean ± SE percentage of body fat for overweight
volunteers taking 2 kind of coffee extracts.

| | Body fat (%) | | | | |
|---|---|---|---|---|---|
| | | | | Start − | |
| | Start | Week 4 | Week 12 | Week 12 | P-value |
| Product A | 27.2 ± 2.0 | 25.6 ± 1.8 | 23.6 ± 1.7 | 3.6 ± 0.3 | P < 0.05 |
| Product B | 26.9 ± 2.1 | 26.7 ± 2.0 | 26.2 ± 2.0 | 0.7 ± 0.4 | NS |

One skilled in the art will appreciate that the present invention can be practical by other than the embodiments described herein, which are presented for purposes of illustration and not of limitation.

We claim:

1. A method of inhibiting human liver glucose-6-phosphatase (Glc-6-Pase) activity comprising the step of administering an amount of green coffee extract obtained from Coffea robusta effective to treat a subject.

2. The method of claim 1 wherein the green coffee extract contains a ratio of 5-caffeoylquinic acid (5-CQA) and total chlorogenic acids (tCGA) (5-CQA/tCGA) between about 0.2 and about 0.3.

3. The method of claim 1 wherein the concentration of total chlorogenic acids (tCGA) in the green coffee extract is greater than about 45%, and the concentration of 5-caffeoylquinic acid (5-CQA) in the green coffee extract is greater than about 10%.

4. The method of claims 1, 2 or 3 wherein the green coffee extract is administered in an amount effective to guarantee a suitable bioavailability of chlorogenic acids (CGA) in humans.

5. The method of claims 1, 2, 3, or 4 wherein the green coffee extract is administered in a dose between about 200 mg and about 1,000 mg per day.

6. The method of claim 5 wherein the dose is about 400 mg per day.

7. A method of reducing glycemia in humans comprising the step of administering an amount of green coffee extract obtained from Coffea robusta effective to treat a subject.

8. The method of claim 7 wherein the green coffee extract contains a ratio of 5-caffeoylquinic acid (5-CQA) and total chlorogenic acids (tCGA) (5-CQA/tCGA) between about 0.2 and about 0.3.

9. The method of claim 8 wherein the concentration of total chlorogenic acids (tCGA) in the green coffee extract is greater than about 45%, and the concentration of 5-caffeoylquinic acid (5-CQA) in the green coffee extract is greater than about 10%.

10. The method of claims 9 wherein the green coffee extract is administered in an amount effective to guarantee a suitable bioavailability of chlorogenic acids (CGA) in humans.

11. The method of claims 10 wherein the green coffee extract is administered in a dose between about 200 mg and about 1,000 mg per day.

12. The method of claim 11 wherein the dose is about 400 mg per day.

13. A method of increasing the Muscle Mass/Fat Mass ratio (MM/FM) in humans comprising the step of administering an amount of green coffee extract obtained from Coffea robusta effective to treat a subject.

14. The method of claim 13 wherein the green coffee extract contains a ratio of 5-caffeoylquinic acid (5-CQA) and total chlorogenic acids (tCGA) (5-CQA/tCGA) between about 0.2 and about 0.3.

15. The method of claim 14 wherein the concentration of total chlorogenic acids (tCGA) in the green coffee extract is greater than about 45%, and the concentration of 5-caffeoylquinic acid (5-CQA) in the green coffee extract is greater than about 10%.

16. The method of claims 15 wherein the green coffee extract is administered in an amount effective to guarantee a suitable bioavailability of chlorogenic acids (CGA) in humans.

17. The method of claims 16 wherein the green coffee extract is administered in a dose between about 200 mg and about 1,000 mg per day.

18. The method of claim 17 wherein the dose is about 400 mg per day.

19. A method of reducing the body weight in humans comprising the step of administering an amount of green coffee extract obtained from Coffea robusta effective to treat a subject.

20. The method of claim 19 wherein the green coffee extract contains a ratio of 5-caffeoylquinic acid (5-CQA) and total chlorogenic acids (tCGA) (5-CQA/tCGA) between about 0.2 and about 0.3.

21. The method of claim 20 wherein the concentration of total chlorogenic acids (tCGA) in the green coffee extract is greater than about 45%, and the concentration of 5-caffeoylquinic acid (5-CQA) in the green coffee extract is greater than about 10%.

22. The method of claims 21 wherein the green coffee extract is administered in an amount effective to guarantee a suitable bioavailability of chlorogenic acids (CGA) in humans.

23. The method of claims 22 wherein the green coffee extract is administered in a dose between about 200 mg and about 1,000 mg per day.

24. The method of claim 23 wherein the dose is about 400 mg per day.

25. A method of reducing the Body Mass Index (BMI) in humans comprising the step of administering an amount of green coffee extract obtained from Coffea robusta effective to treat a subject.

26. The method of claim 25 wherein the green coffee extract contains a ratio of 5-caffeoylquinic acid (5-CQA) and total chlorogenic acids (tCGA) (5-CQA/tCGA) between about 0.2 and about 0.3.

27. The method of claim 26 wherein the concentration of total chlorogenic acids (tCGA) in the green coffee extract is greater than about 45%, and the concentration of 5-caffeoylquinic acid (5-CQA) in the green coffee extract is greater than about 10%.

28. The method of claims 27 wherein the green coffee extract is administered in an amount effective to guarantee a suitable bioavailability of chlorogenic acids (CGA) in humans.

29. The method of claims 28 wherein the green coffee extract is administered in a dose between about 200 mg and about 1,000 mg per day.

30. The method of claim 29 wherein the dose is about 400 mg per day.
